(19) United States
(12) Patent Application Publication
(10) Pub. No.: US 2009/0243833 A1
(43) Pub. Date: Oct. 1, 2009

(54) MONITORING SYSTEM AND METHOD FOR PATIENT CARE

(76) Inventors: Ching Ching Huang, Glendora, CA (US); Franklin D. Hwang, Glendora, CA (US); Jennnifer Peng, Huntington Beach, CA (US); Francis N. Hwang, New York, NY (US); Francine N. Hwang, Rancho Palos Verdes, CA (US)

Correspondence Address: Ching Ching HUANG, 949 EAST Plymouth Street, Glendora, CA, 91740, US

(21) Appl. No.: 12/383,536

(22) Filed: Mar. 25, 2009

Related U.S. Application Data
(60) Provisional application No. 61/072,262, filed on Mar. 31, 2008.

(51) Int. Cl. G08B 26/00; G06Q 50/00; H04B 1/38

(52) U.S. Cl. 340/505; 705/3; 455/90.1

(57) ABSTRACT

A patient care monitoring system and method employ active RFID devices integrated with digital processing, memory and timing circuitry for patient identification, care giver identification and for identification of each prescribed treatment, procedure, medication and general and/or special care action. At the point-of-care, each care action identity device will match directly with the targeted patient identity device or issue an error warning to prevent mistakes. The patient identity device will also interact with an associated sensor network to proactively prompt care givers to provide general care actions, such as altering a patient's laying position, changing bed pan/clothing/bed sheet, etc. for invalid patients. Also the patient identity tag will furnish periodic records of every care action, mistakes, remedies, care givers' identities and time and date for a central processor of a healthcare facility to monitor the quality of patient care. Such record can also be potentially accessed via the Internet by the responsible regulatory agencies, accreditation associations, insurance firms and even patients' families to ensure patient care is meeting the standards as well as medical billing accuracy.

### RELATED APPLICATIONS

This non-provisional application claims the benefit of U.S. Provisional Patent Application No. 61/072,262 filed on Mar. 31, 2008 and the non-provisional patent application Ser. No. 12/217,415 filed on Jul. 3, 2008.

### FIELD OF THE INVENTION

This invention relates to the patient care monitoring system, associated method and its constituent devices which will provide monitoring, proactive prompts for treatment, recording and reporting of all prescribed actions as well as general care actions, mistakes and corrective measures administered for each patient. The patient care monitoring system matches the identification of the patient to their corresponding prescribed daily treatments, procedures, medications and general care. The system also matches the time frame specified for each of these care actions with the corresponding patient. When a mismatch is detected, the system will sound an alarm, and/or activate a warning display, and prompt any healthcare worker within its radio frequency transmission range to correct the mistake. The system will also sound an alarm or activate a warning display when the prescribed action is not acted upon or corrected within its specified time frame.

The system will further record and report prescribed treatment, procedure and medication given to a patient throughout the day along with the time of the care action. The system identifies, records and reports which healthcare worker was administering the care action as well as any mistakes and subsequent corrective actions.

### BACKGROUND OF THE INVENTION

To err is human. However, medical errors, according to many research studies, have caused on average some 195,000 deaths in the U.S. annually. These deaths are preventable. The most common type of preventable medical errors are: incorrect administering of drugs (wrong prescription, wrong dosage, given to wrong patient and at wrong time), hospital acquired infections (unclean or improperly cleaned hands of healthcare staff, improperly sterilized equipment), postoperative bloodstream infection (un-sterilized and/or improper handling of sterile equipment, unclean hands), ventilator-associated pneumonia (again, un-sterilized and/or improper handling of sterile equipment, unclean hands) and negligence in basic cares (bed sores, falls, dehydration, malnutrition, etc.). The estimated cost for these medical errors is between $8.5 to $14.5 billion dollars annually. In the current climate of ever escalating healthcare costs, to prevent and reduce medical errors have become an absolute necessity. There is also a moral responsibility to provide quality healthcare to patients.

Medicare patients (65 years and older) account for 45% of all hospital admissions (excluding obstetric patient) in the U.S. This population suffers much more severe consequences from medical errors due to declining health, decreased immunological resistance and decreased recuperative ability. Consequently, out of the average 195,000 preventable deaths due to medical errors annually, a disproportional number of patients are elderly.

The latest statistics on U.S. nursing homes stated that there are 1.6 million patients occupying 1.9 million available beds, and the average stay of patients being discharged is over 290 days. For those not being discharged the average stay of patients is over 800 days. This is a clear indication that most patients in nursing homes as well as increasingly in the hospitals are aged and invalid patients (needless to add, many have difficulty in communicating their needs to healthcare staff).

These aged and invalid patients require additional care such as feeding, changing of bed pans, washing, turning them on their sides periodically, or simply communicating with them. Although each hospital and nursing home has stringent guidelines in how to take care of this type of patient properly, the workload pressure and shortage of nursing staff frequently result in lengthy improper care and further deterioration of the patient's health status. The lack of proper care thus costs the entire healthcare system (patients, their families, taxpayers, insurance companies) much more money, suffering and, in the worst case, unnecessary deaths.

It is not unusual for a person to observe the foul odor in a hospital wing or nursing home housing mostly aged and invalid patients. Numerous complaints have come from families that the patients frequently have severe skin rashes, lesions and bed sores to the degree of rotting flesh. All these are clear signs that proper patient care are not provided by these healthcare facilities.

On the other hand, by visiting any hospital or nursing home admission office, one will be bombarded with how well they have cared for their patients as well as shown the reams of patient care guidelines that they adhere to and the records of their adherence. However, there is no unbiased monitoring system that can provide data on: how often each patient is cared for, the percentage of properly carrying out treatment, procedures and medications prescribed by physicians on time and on specification other than what is recorded by nurses or their aids.

Several U.S. Congressional hearings and subsequent laws and regulations had resulted in the establishment of Federal Minimum Standards for nursing care facilities. Furthermore, each state also sets forth their minimum standards. However, the lack of effective monitoring methods and systems in providing realistic patient care monitoring data is a huge handicap in enforcing the laws and regulations particularly on those facilities supported principally by the Medicare and Medicaid programs.

Besides medical errors and negligence in providing necessary care actions, another aspect is fraudulent billing, i.e. charges without actually delivery of medical care actions, by not only healthcare facilities, but also increasingly by home care providers. Since the federal government medical insurance (Medicare) and the states' assistances are the biggest payers, they suffer the most financial loss.

Here we put forward an invention consisting of a method, monitoring devices and a system that does not disrupt the existing work routine of a healthcare facility and does not add any additional work step to the care giver. This system also ensures proper patient care is registered and reported on a daily or periodic basis. This data certainly can be forwarded to the regulatory agencies as well as family members of the patients to ensure proper care is continuously provided to those unfortunately sick, aged and/or invalid on a daily or periodic basis instead of just the period prior to or after an inspection by regulatory agencies. Furthermore, by logging these care actions, it provides a mean to track the accuracy of billing by insurance payers and thus reducing fraud.

There are numerous prior arts as cited in the Reference Section detailing various patient care monitoring systems and methods. All of them require special adaptations in order to achieve some measure of monitoring patient care. Therefore, not only new procedures must be adopted by a healthcare facility, but also added work steps. For example, added work steps such as: scanning the patient identification band, scanning every treatment/medication identification tag, waiting for remote processors to give an O.K. before proceeding in carrying out the care action, will greatly disrupt the work flow and reduce efficiency. Many of the basic care actions, such as changing a bed pan, bathing, altering a patient's laying position, special diet, etc., are not necessarily codified in most healthcare facilities, other than written in the patient's chart. Therefore, the actions are not monitored or tracked and are ignored in all the prior arts. Furthermore, many of the care actions, prescribed and general, have a timing element associated, such as medications, physiological measurements, altering a patient's laying position. Consequently, the patient care monitoring system must be able not only to record the timing of a care action being executed, but also proactively prompt the care giver to provide the care action within a specified time frame. Again, this aspect has been missing in the prior arts.

During a standard patient admission process into a healthcare facility, he/she is assigned an identification wrist band (such as a simple printed label with information like name, age, gender to assignment to a specific department/hospital wing and a specific patient room), which will stay with the patient for his/her entire stay in the facility along with a patient chart as well as entry of informational data into the central computer of the facility. During the patient's stay in the facility, a physician or attending care giver will typically examine the patient periodically (daily in hospital) and prescribe specific care actions to the said patient. The daily prescribed care action corresponding to a specific patient is entered into the patient chart as well as into the central computer of the care facility. Furthermore, standard general care actions, such as changing the patient's laying position and bed pans periodically for invalid or aging patients, bathing patients and diet precautions, etc., are also included (automatically or manually by the care giver) into the care instruction set for each patient.

To identify each patient and the treatments, procedures, medications and care actions prescribed to each patient, many prior arts suggested various approaches other than simple printed label, such as adding bar code, magnetic strip, Infrared (IR) pattern or radio frequency identification device (RFID) to the identification wrist band and to the label attaching to each care action delivery agent, administering devices or paper work as a mean in matching the patient with the care action-prescribed to him/her. U.S. Pat. Nos. 4,857,713 (Brown) and 4,857,716 (Gombrich, et al.) use printed bar code method for patient and care action identifications. Proper patient care monitoring is accomplished by scanning the bar codes of the patient and care action label as well as having a linked processor to conduct the matching. U.S. Pat. Nos. 6,824,052, 6,830,180 and 6,910,626 (Walsh) expanded the identification method to not only printed bar code, but also magnetic strip and/or Infrared (IR) pattern. As mentioned before, these methods and systems create added work steps for typical healthcare facilities as well as new equipment, linkage and installation. Also, the chaos/confusion will occur from the inaccuracy of scanning a bar code, swiping magnetic cards through a reader or line-of-sight requirements to do IR pattern recognition (error rate between 5 to 10%). U.S. Pat. Nos. 5,071,168 and 5,381,487 (Shamos) employ personal characteristics (such as fingerprint, eyeprint, and footprint) as patient identification code. Treatment/care action will only be given based on matched patient identification code. This is an even more tedious and time consuming method of patient identification. Many inaccuracies will result from the arbitrary selection of matching confidence level.

The RFID approach requires less effort of a care giver to read the identification code of a patient or a treatment/care action label/tag, since it only demands proximity to the reader and without the stringent line-of-sight demanded by optical scanner (bar code and IR methods) or moving the identification band/tag through a contact magnetic strip reader. However, a passive RFID as presented in the U.S. Pat. Nos. 6,671,563 and 6,915,170 (Engleson, et al.) still requires a reader to be placed close to the patient's identification band and to the treatment/care action tag in order to obtain the identification codes. This approach is more suitable for identification of objects rather than persons. The added work steps (placing the reader close to the identification band/label/tag and check whether a reading is made) to accomplish this data acquisition will disrupt the heavy work load of healthcare workers and result in frequent-non-usage.

Other prior arts, such as U.S. Pat. No. 7,384,410 (Eggers, et al.), use RFID method to identify patients and care delivery devices to achieve error avoidance. However, this approach will not monitor many of the care actions that require no administering devices.

The system and method stipulated in the U.S. Pat. Nos. 5,883,576, 6,255,951 and 6,346,886 (De La Huerga) as well as U.S. Pat. Nos. 6,961,000, 7,158,030 and 7,382,255 (Chung) employs the approach of reading and sending the identification codes from the patient and the treatment/care action device along with a relational check code (in Chung's patents) to a separate and independent processor for matching to determine the action to be executed corresponds to the patient. A display and alarm will then inform the care giver whether a mismatch exists. This multiple-element system not only produces added work steps (scanning/reading of the identification devices and waiting for direction from the processor), thus discouraging usage by care givers and adoption by healthcare facilities, but will also not monitor those required care actions, such as bathing invalid patients, changing wet clothes, changing bed pan, rotating patient laying/sitting posture, etc., that do not carry identification labels/tags.

The invention presented here will employ active RFID technique (contains a power source to transmit and receive RF signals for transmitting its stored codes and for receiving external data) in the patient and treatment/care action identification. This approach will provide direct and immediate verification between the patient identification band and the treatment/care action ID tag. The healthcare worker does not take any extra step to facilitate the reading of the RFID tags, thus ensuring the usage of this invention. Active RFID also achieves the determination of a match or mismatch prior to administering care action at the point-of-care. The patient ID band will also (through communication with other sensors) determine whether other general care actions without ID tags have been executed within the prescribed time frame. Furthermore, it will interact with the care giver's identification tag/band to proactively prompt him/her to provide the required care actions as well as record all the care actions given with respect to time and correctness along with the identities of the care givers administered all the care actions.

### SUMMARY OF THE INVENTION

Conforming to the standard practice of a hospital or nursing home, this invention presents a patient care monitoring system and method that employs active RFID integrated with a digital processor as a device (ID band or ID tag) to transmit the programmed identification codes for each patient, care giver and for each treatment, procedure, medication and care action. By having each identification device capable of receiving and deciphering only the signals containing its own unique identification code, the patient identification wrist band will thus determine whether the treatment/procedure/medication/care action label/tag presented to him/her at the point-of-care matches the one prescribed by his/her physician. Equally, the treatment/care action label/tag will match the received patient ID code to its assigned patient code to determine whether it is the correct patient. If there is a mismatch, then a visual or audio alarm integrated into the identification devices will be displayed and/or sounded to alert the care giver of the error. Since the standard routine in a healthcare facility is for an attending physician to examine his/her patient in the morning and entering prescribed care action for the day into the patient's chart and the facility's computer system (typically at the terminals in a nurse station), the present invention will translate the prescriptions into corresponding treatment/procedure/medication/care action codes within its central processor and transmit the daily care actions and schedule via wireless communication through a RF transceiving device within each patient's room to each corresponding patient's identification band. At the same time, the central processor will send the prescribed treatments, procedures, medications and care actions to appropriate departments of the facility to program an active RFID identification tag with the unique code corresponding to the treatment, procedure, medication or care action along with the targeted patient identification code. These ID tags will then be attached to the care delivery device and/or paper work to be presented to the patient at the point-of-care. Each patient ID band and the care action ID tag will interact with each other and cross check with each other to ensure they correspond to each other before the care action is administered. At the time of administering, both the patient ID band and the care action ID tag will record the event and time as well as the ID code of the care giver. The patient ID band can also receive input from other measuring sensors, such as posture position, wetness, body temperature, pulse/heart rate to determine whether an alert to the care giver should be generated. Also, if a prescribed or general care action at a specific time frame was not administered, then, the patient ID band will transmit an alert signal continuously to prompt any care giver to provide the care action as soon as possible. All the care actions administered or non-conformance to the prescription or general care guidelines will be recorded by the patient ID band and transfer through the same RF transceiver device to the central processor to report and alert the quality control personnel of the facility. At the same time, all the treatment/care action ID labels/tags will be returned to the corresponding departments after their usage to download the recorded data and transfer to the central processor. After downloading, the memory of each ID tag can be cleared and reprogrammed for reuse.

### BRIEF DESCRIPTION OF THE DRAWINGS

FIG. 1 illustrates a wrist band configuration of a patient's identification device where [1] indicates the housing for battery pack, [2] is where the RF transceiver and digital processing, memory and timing circuitry is housed, which links to [3] the capsule for an antenna. FIG. 2 shows a version where it can be the identification device worn by the healthcare giver with [7] housing the battery pack, [9] being a combination of a watch module and the RF transceiver/processing/memory/timing circuitry, and [10] being the capsule for an antenna and visual display.

FIG. 3 demonstrates a treatment/procedure/medication/care action tag, which is attached to a medicine delivery vessel [13]. In this drawing, [11] is an encapsulation of a RF transceiver, digital processing, memory and timing circuitry along with battery pack and an antenna. This waterproof capsule is covered with a printed label stating what the care action is and the patient's name, room number along with other relevant information. The entire capsule is adhered to a disposable tape [12] which in term adheres to a care action administering device [13] (such as a medication dispensing vessel in this drawing), or to the treatment/procedure/care action paper work carried by the care giver to the point-of care. The green [15] and red [16] LED indicators on each care action tag will flash when there is a match or mismatch between the prescribed care action and the patient's identity.

FIG. 4 shows a programmer machine for programming a prescribed treatment/procedure/medication/care action identification tag. This device is linked to the central processor of the healthcare facility to download the prescribed care action into a care action identification tag. Care action in code will be programmed into the inserted tag along with the targeted patient identification code. The programmed data will also be shown on the display screen [17], and an integral printer [19] will print out the coded care action, patient name, identification characteristics and room number on a visually readable label [20] that will automatically be attached to the care action identification tag. Manual programming can be done through key pads [18, 21 and 22].

FIG. 5 illustrates how an attending physician per hospital work routine will enter his/her prescribed treatments, procedures, medications and/or care actions as well as timing for a specific patient after a round of examination into the central processor of the healthcare facility. The prescription will then be translated into corresponding care action codes and forwarded to responsible departments to program the care action identification tags and for administering.

At the same time, the physicians' prescriptions will also be sent via a RF transceiving device in a patient room [27] to the patient identification band [23] as shown in FIG. 6. FIG. 7 demonstrates that the patient's identification band will interact with each care action identification tag [26] being administered to assure it matches the prescription and timing. The patient identification band [23] will record all the care actions administered throughout the day, their correctness and timing. The record will be transferred from the patient band [23] to the central processor again through the [27] RF transceiving device on daily basis for the central processor to produce a patient care monitoring report as indicated in FIG. 7.

FIG. 8 presents one type of sensor network that enables the invention to provide proactive prompts to care givers for prescribed care action or general required care action. In this illustration a thin and flexible pad [27] consists of a network of pressure transducers [29], which will be placed underneath the patient's bed sheet. The signals from various pressure transducers will indicate the body movement (or lack of) of a patient as a function of time. For an invalid patient, this pad will signal the patient identification band to prompt any care giver walking into the patient room to alter the patient's body position when required to prevent and eliminate bed sores. Equally, a wetness sensor added to the pad can prompt the care giver to change the bed pan, clothing and bed sheets for the patient.

FIG. 9a provides a block diagram and interaction between the various components of the invention, whereas the prescribed care actions are entered into the central computer of a healthcare facility and transmitted through its intranet to the in-patient-room RF Transceiving Device. This relays to the corresponding patient's ID band and to the Care Action ID Tag Programming Device for programming into a Care Action ID along with the targeted patient ID code. FIG. 9b illustrates the wireless interaction between the patient ID band and a care action ID tag to assure correctness prior to the administering of the care action. FIG. 9c demonstrates that the patient ID band will proactively prompt the care giver's ID band to furnish needed care action per its sensor network inputs or query from its own stored prescribed care action program.

FIG. 10 presents a possible daily patient care quality monitoring report generated by this patient care monitoring system and method.

### DETAILED DESCRIPTION OF THE INVENTION

This invention presents a practical and accurate system to monitor patient care to avoid most common medical errors in a healthcare facility while it adheres to the standard healthcare work procedures and routines in administering patient care. The transparency in conducting the monitoring without requiring care givers to perform additional work steps or disrupting the trust between patients and care givers ensures this invention to be adopted and accepted by healthcare facilities. It also differentiates itself from any prior arts.

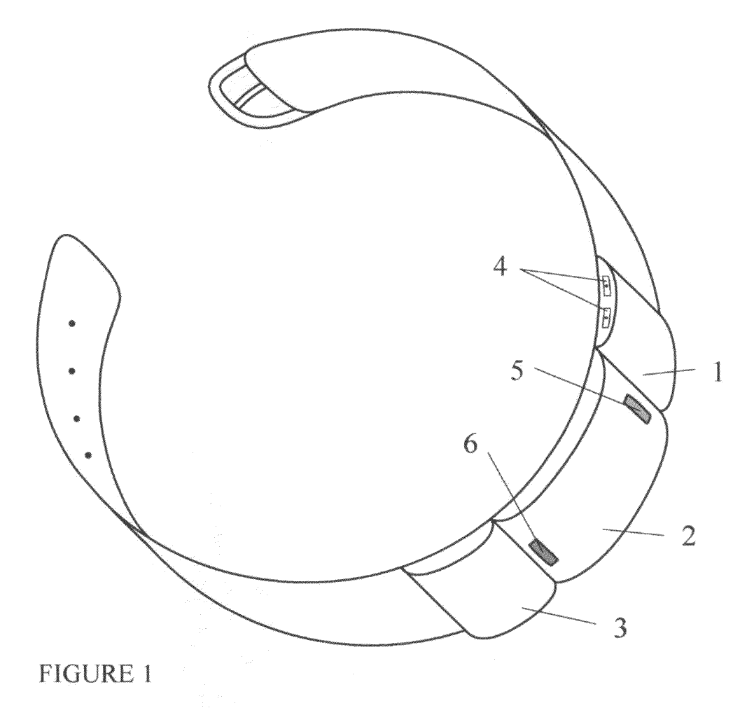

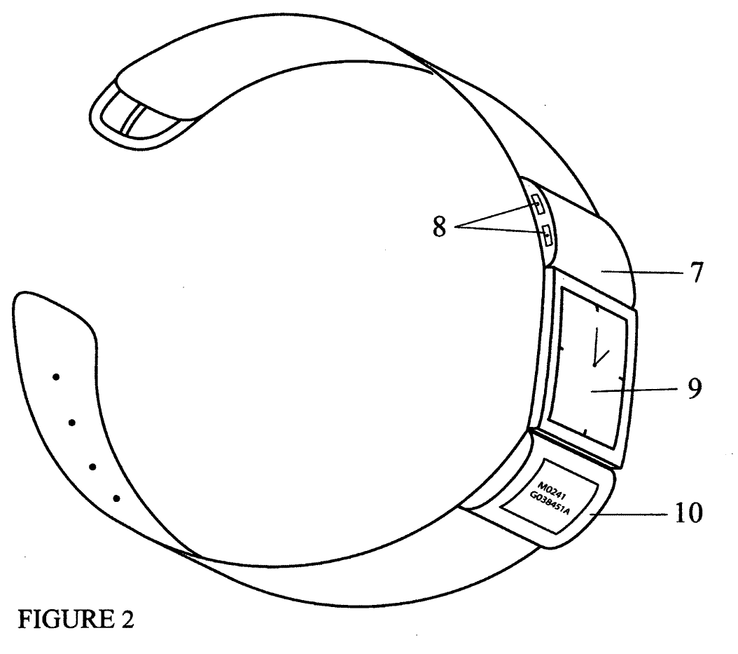

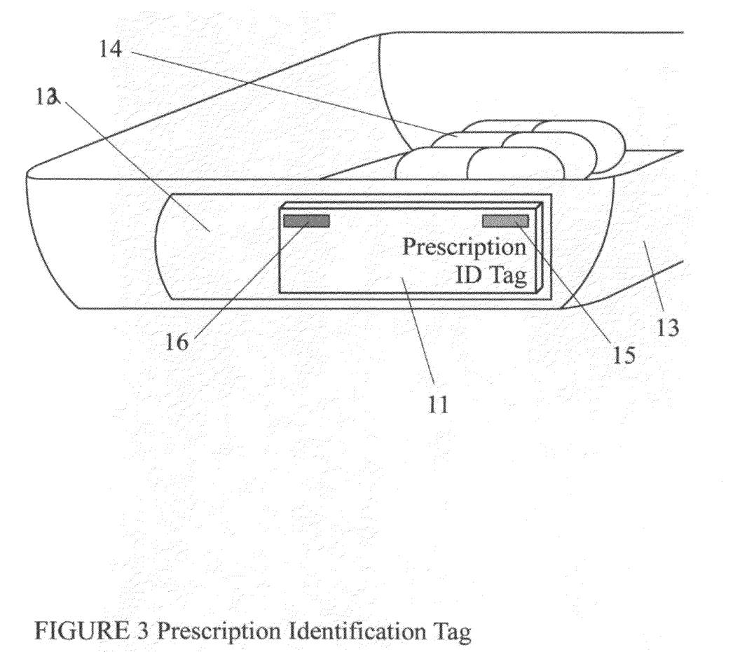

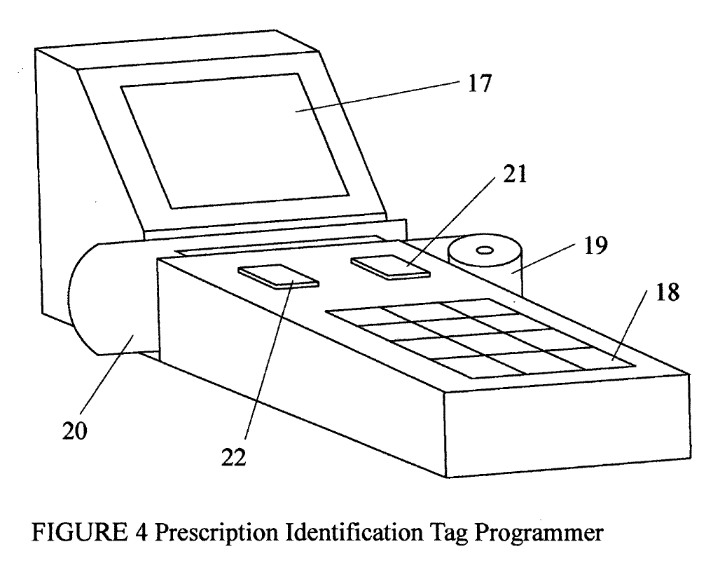

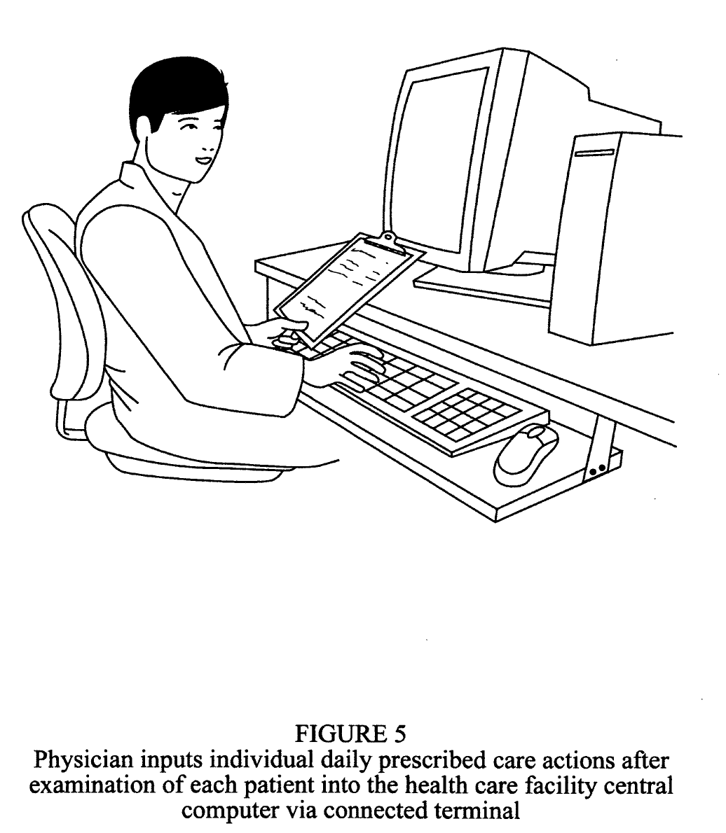

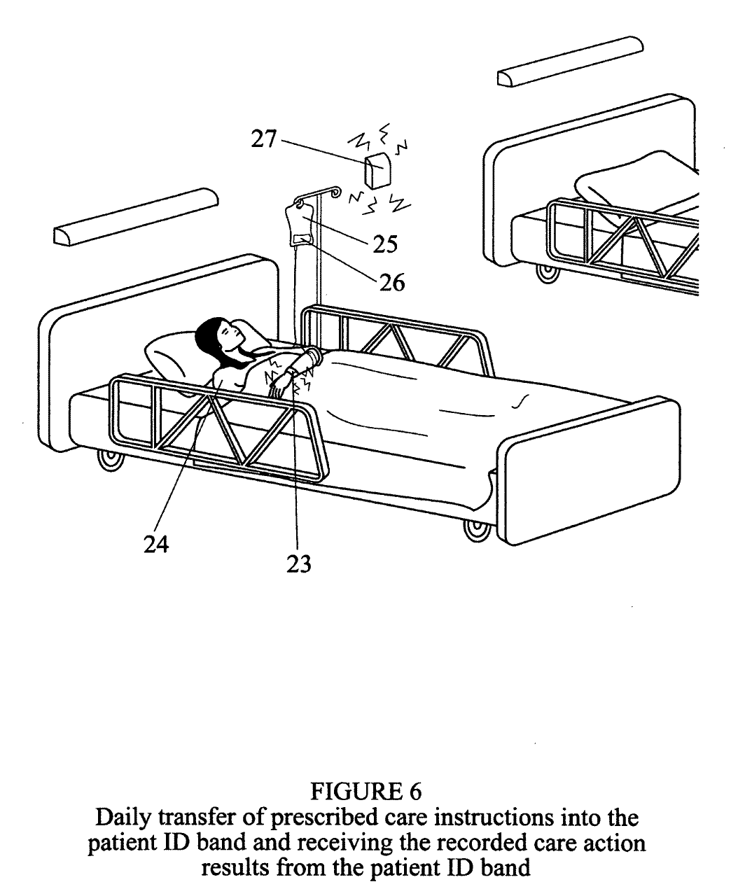

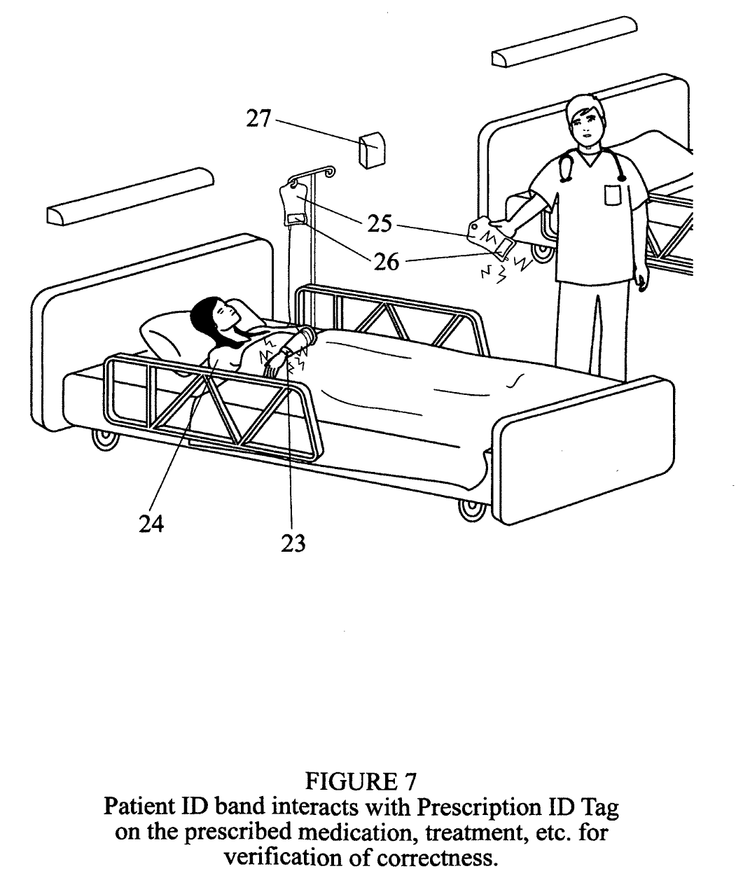

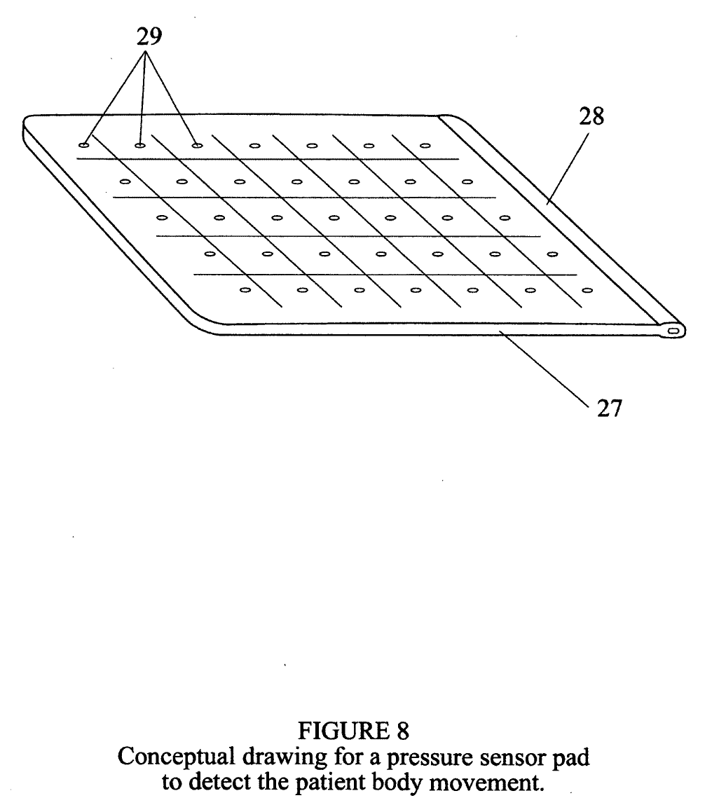

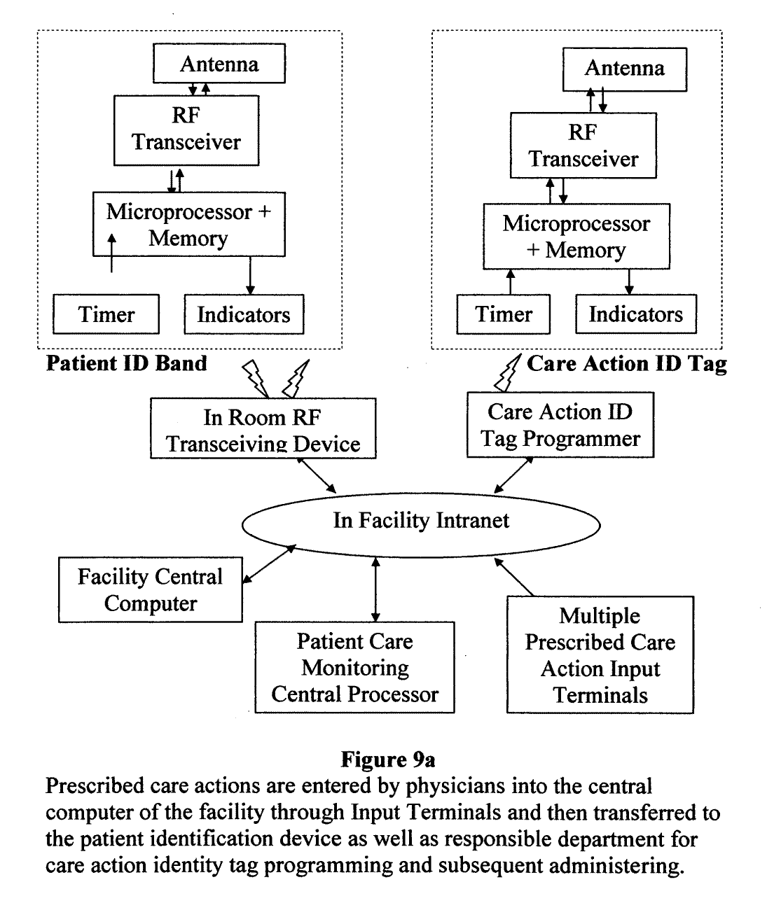

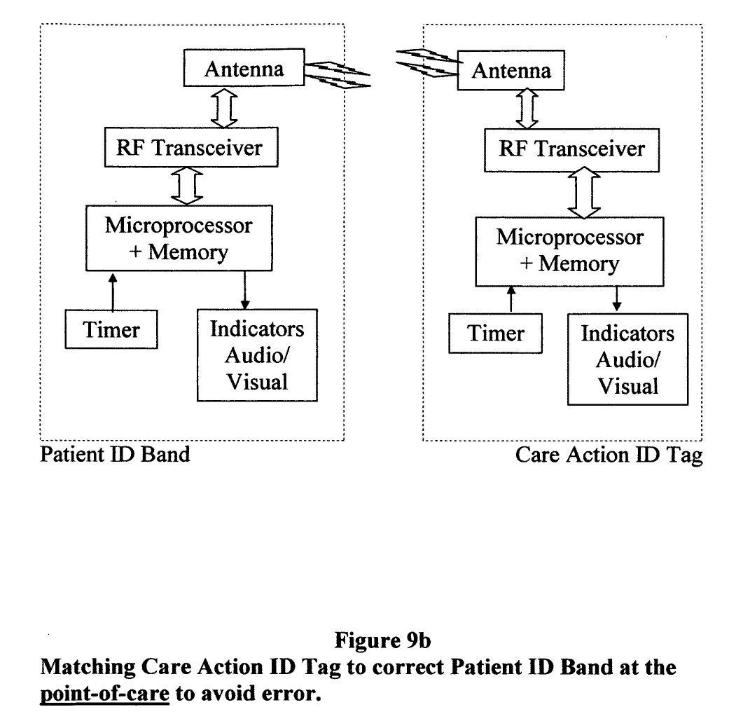

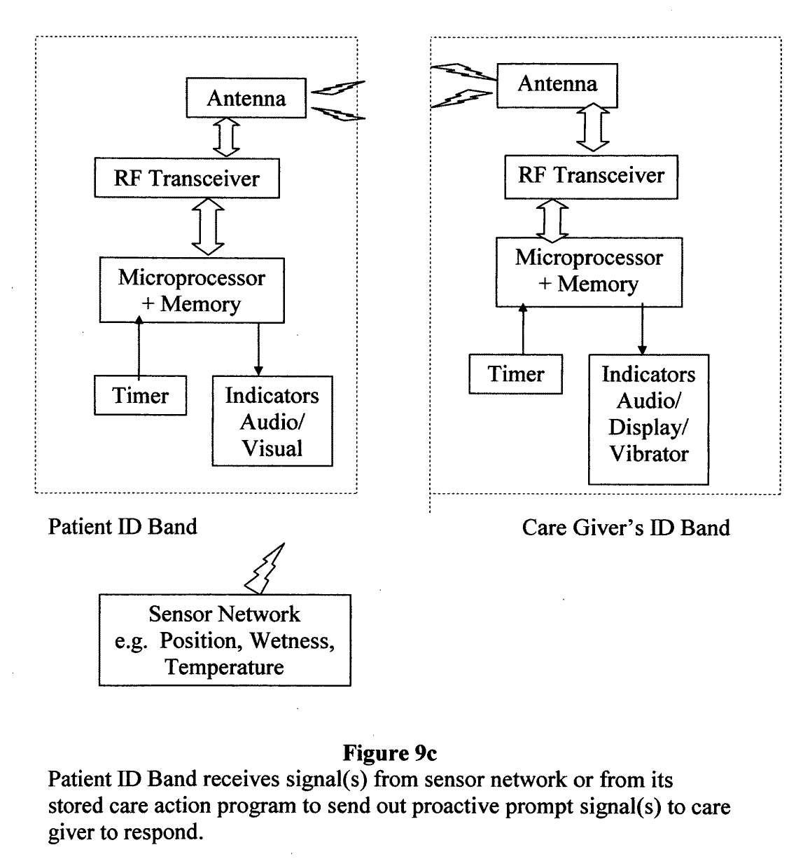

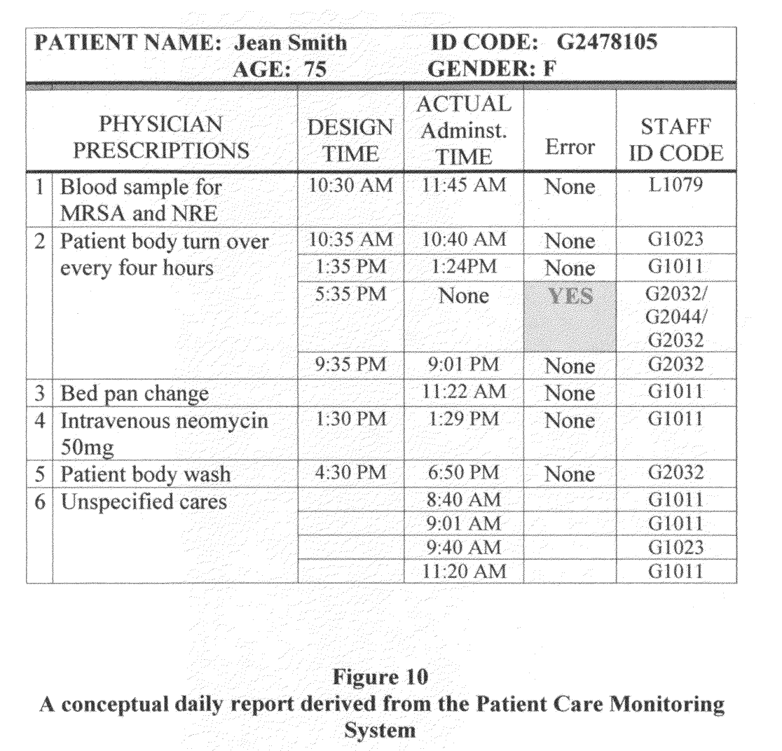

The hardware and software detailed in claim 1 consists of the following hardware components along with imbedded operating software to enable each to function as described below:
1. The patient identification device as illustrated in FIG. 1 is in the most commonly employed configuration of a wrist band. This waterproof wrist band contains a battery pack [1] which can be charged via electrical contacts [4] or electromagnetically without electrical contacts, a central plastic housing [2] for the RF transceiving, digital processing, memory and timing circuitry and a separate plastic capsule [3] for an antenna. With the current integrated circuitries and micro-electronics, all three components can be integrated into a single small housing of 0.5 in (Width)×1.0 in (Length)×0.25 in (Height) or even smaller in size. At the admission of a patient, the admission personnel will enter the relevant patient information, such as name, gender, age, ethnicity, possible illness, physician name(s), hospital room assigned, etc. into the central processor (computer) of the healthcare facility along with generating a unique identification code for the patient. This code will stay with this patient until his/her discharge. The central processor will in turn program a wrist band (as illustrated in FIG. 4—note: the patient identification wrist band and the prescription care action identification tag programming can be done on a same device linking to the central processor) with this assigned patient code and print the patient information on a label to insert into the transparent pocket on top of the wrist band. The admission personnel will then fasten the identification wrist band on the patient's wrist (or ankle) which will be secured for the duration of the patient's stay. The patient ID band will be continuously in receiving mode to receive RF signals. Upon receiving a RF signal tuned to its receiving frequency, such as 2.3 GHz, it will examine the signal string for its own unique identification code. If the code does not exist in the signal string, then it will ignore the signal. If the code does exist, then it will match its stored care action program codes with the care action code in the signal. If it matches, then it will broadcast an “O.K” signal along with its identification code and flash its green LED indicator [5] in FIG. 1 for a period of time. If there is no match in the care action code between its stored program and that from the received signal, then it will transmit a “Mistake” signal along with its identification code and flash its red LED indicator [6] for a period of time. The patient ID band will also transmit a specific prompt signal along with its ID code to alert care giver to correct any mistake or administer the prescribed care action before the specified time period expires. All the signals transmitted by the patient ID band will be in low power range (a few milliwatts) to achieve a short distance (3-10 ft) receiving by other identification devices within a patient room. The patient ID band will record all these interactions and time and date and transmit the record to the central processor of the healthcare facility on daily basis.
2. The care giver identification device as illustrated in FIG. 2 is in a configuration of a fashionable wrist watch. This waterproof wrist watch contains a battery pack [7], which can be charged via the contacts [8] or electromagnetically without electrical contacts, a central housing [9] for the RF transceiving, digital processing, memory and timing circuitry along with the watch mechanism and a separate plastic capsule [10] for antenna and a display module. This care giver identification wrist watch will contain a unique code assigned to each individual worker during his/her employment in the facility. This care giver ID device will transmit its identification code continuously in burst mode (such as once every second or every other second) and, in between the transmissions, it will receive any RF prompt signals from the patient ID bands and activate its display [10] to show the nature of the prompt on care action not executed or mistake on care action to be administered as well as starting its built-in vibration device to alert the care giver.
3. Identification device in the configuration of a label or tag for prescribed treatment, procedure, medication and any special care action, as shown [11] in FIG. 3, is virtually identical to the patient identification device in terms of RF transceiving, digital processing, memory and timing circuitry except all of them along with battery pack and antenna are contained in a single sealed plastic housing of 0.5 in (Width)×1.0 in (Length)×0.25 in (Height) or even smaller in size. This type of tag will each be programmed by the programming device, shown in FIG. 4, with the code of a particular prescribed treatment, procedure, medication or care action along with the identification code of the targeted patient. This care action tag will continuously transmit, in burst mode, a signal containing its programmed care action code and the corresponding patient ID code at a cycle of once every second or some other frequency rate. The transmission will be at a specific frequency, such as 2.3 GHz, and at a low power, typically in a few milliwatts range, to affect a short distance signal transmission (3 to 10 ft range). In between transmission, the care action tag will be in receiving mode to receive signals from the patient ID band. It will ignore any signal that does not have the correct patient ID code that it carries in corresponding to the care action code. If an “O.K.” signal is received with correct patient code, then it will flash its green LED indicator [15] to signal match has been verified. When a “Mistake” signal is received with correct patient code, then it will flash its red LED indicator [16] and/or audio warning tone to signal error.
4. A central processor can be the central computer of a healthcare facility or it can be a separate personal computer (PC), a server or a combination of multiple PC and servers, which is linked with the central computer of a healthcare facility via intranet such as a wired or wireless large area network (LAN) or wide area network (WAN). This central processor will take the prescriptions issued by attending physicians (typically each morning after their rounds of examination of patients as illustrated by FIG. 5) and convert them into alpha-numerical codes corresponding to the specific treatments, procedures, medications (type and dosage) and special care actions along with the identification codes of the targeted patients as well as time frame to be administered. These coded data along with prescriptions entered by the physicians will be transmitted via intranet to each responsible department and/or nursing station to program and prepare the care action tags as well as administering schedule as illustrated in FIG. 9a. This central processor will also transmit these coded prescribed care actions and time schedule to the corresponding patient's ID band via RF transceiving device, [27] of FIG. 6, located in each patient room as shown in interaction block diagram of FIG. 9a. The same transceiving device [27] will also relay the daily care administering log recorded by a patient ID band back to the central processor for report presentation and data archiving.
5. A RF transceiving device, [27] of FIG. 6, which is linked to the central processor through intranet (e.g. an Ethernet connection) and contains a RF transceiving and digital processing circuitry along with antenna to convert the data strings received from the central processor and to transmit them via RF to the patient ID bands located within the room that this device [27] is located. It will also receive the daily care administering log from the patient ID bands located within a room via RF and convert them into proper format/protocol (such as TCP/IP) for transmission via intranet to the central processor. FIG. 6 illustrates the transmission and receiving actions taking place between this device [27] and the patient's ID band [23] worn by a specific patient [24].
6. A care action identification tag programming machine, shown in FIG. 4, which programs the memory of a care action identification tag placed within it with a set of code corresponding to the type of care action, dosage (in term of medication), delivery mean and time frame for the administering along with the patient's identification code that this care action is prescribed to. It will concurrently print out a readable label [19, 20] adhering to the care identification tag for ease and correct delivery to the right patient room and to the right patient. This machine will be used in each department and nurse station of the healthcare facility and is linked to the central processor through intranet for downloading the care action identification codes and corresponding patient's identification code that the department and/or nurse station will be responsible to execute.
7. When a care action delivery device/agent, [25] of FIG. 7, or associated paper work is brought to a patient, the care action identification tag [26] attached to this delivery device/agent or paper work will transmits its stored codes and associated patient's identification code continuously. FIG. 7 shows that the care action tag [26] attached to an intravenous medication bag [25] performing this process. Upon receiving this signal string, the patient's ID band [23] will examine whether its unique identification code is within the signal string. If it is not, then the patient ID band will ignore the signal string. If it is, then the ID band will further examine whether the care action codes match those stored in its memory as part of the care action program prescribed by his/her physician for the day. If it matches, then the ID band will transmit an “O.K.” signal along with its own identification code. Otherwise, it will send a “Mistake” signal with its own identification code. For “O.K.” status, the ID band will also flash the green LED [5] of FIG. 1, for a period of time. Red LED [6] will be flashed when “Mistake” status is determined (audio alarm can also be included in the warning) along with sending out a warning signal to trigger the vibration mode of the care giver's identification band/tag to prompt the stop of administering and examine the mistake. The care action identification tag, upon receiving either the “O.K.” or “Mistake” signal with correct corresponding patient identification code from the patient ID band, will activate the flashing of green LED [15] or red LED [16] and/or audio warning on its housing as presented in FIG. 3. All these interactions described in this section occurring at the point-of-care are illustrated by the block diagram in FIG. 9b and are immediate as well as transparent to the care giver except when a mistake warning or no indicator/warning (signaling the patient ID code does not match the patient ID code included in the care action tag) happens.
8. The patient ID band will also periodically examine its stored care action program vs. time to determine whether a prescribed action has been administered. If not, then the ID band will issue a prompt signal which can activate the display and vibration of a care giver's identification band/tag [10] in FIG. 2 and/or transmitted through the RF transceiving device [27] in FIG. 6 to the central processor for displaying alert status in the nursing station responsible for the patient.
9. The patient ID band will also receives signals from a patient monitoring, sensor network, such as from a pressure transducer pad (as show in FIG. 8), wetness sensor, pulse/oximetry sensors and/or heart rate sensors to determine whether specific general care action, such as changing the patient's laying position to prevent bed sores, or changing bed pan, changing clothing or bed sheets is required. If the need is there, then the ID band will issue prompt signals to activate the display and vibration of the identification band/tag [10] of any care giver within his/her room as well as transmit through the RF transceiving device [27] to the central processor to display an alert to the care givers in the nursing station responsible for the patient.
10. The patient ID band will also record all the care action administered and time and date as well as verify all the prompts and resulting actions in its memory. At a designated time, it will transmit this log through the RF transceiving device [27] to the central processor for it to process into a daily or periodic patient care monitoring report as demonstrated in FIG. 9a and FIG. 10.
11. The care action identification tag will be returned to the appropriate department after administering for battery charging, disinfecting and reuse (clear codes in its memory and reprogram with a new set of instruction codes).
12. An electromagnetic (non-electrical contact) battery charger can be placed close to the patient ID band to fully charge the band's internal battery pack.
Current U.S. Class: 235/437, 472.02; 340/572.1, 573.1, 573.7, 604, 614, 669; 700/108, 109, 226; 705/2, 3, 9
Current International Class: G06F 11/30, 19/00; G06K 5/00, 7/10; G08B 21/02, 04, 20; G08B 25/10, 29/18, 31/00
Field of Search: 235/380, 470, 437, 462.01-.09, .34, .46, 472.02; 340/572.1, 573.1, 573.7, 604, 614, 669; 604/67; 700/108, 109, 226; 705/2, 3, 9, 17; 713/189; 714/752

Reference Cited
Related U.S. Patent Documents

| | | |
|---|---|---|
| 4,857,713 | Aug. 15, 1989 | Brown |
| 4,857,716 | Aug. 15, 1989 | Gombrich, et al. |
| 5,071,168 | Dec. 10, 1991 | Shamos |
| 5,381,487 | Jan. 10, 1995 | Shamos |
| 5,760,704 | Jun. 2, 1998 | Barton, et al. |
| 5,883,576 | Mar. 16, 1999 | De La Huerga |
| 6,139,495 | Oct. 31, 2000 | De La Huerga |
| 6,255,951 | Jul. 3, 2001 | De La Huerga |
| 6,346,886 | Feb. 12, 2002 | De La Huerga |
| 6,671,563 | Dec. 30, 2003 | Engelson, et al. |
| 6,824,052 | Nov. 30, 2004 | Walsh |
| 6,830,180 | Dec. 14, 2004 | Walsh |
| 6,910,626 | Jun. 28, 2005 | Walsh |
| 6,915,170 | Jul. 5, 2005 | Engleson, et al. |
| 6,961,000 | Nov. 1, 2005 | Chung |
| 7,158,030 | Jan. 2, 2007 | Chung |
| 7,382,255 | Jun. 3, 2008 | Chung |
| 7,384,410 | Jun. 10, 2008 | Eggers, et al. |
| 7,388,497 | Jun. 17, 2008 | Corbett, et al. |
| 7,413,544 | Aug. 19, 2008 | Kerr, II |
| 7,447,644 | Nov. 4, 2008 | Brandt, et al. |
| 7,448,996 | Nov. 11, 2008 | Khanuja, et al. |

Foreign Patent Documents

| | | |
|---|---|---|
| WO/2003/107252 | Jun. 17, 2003 | Klass, et al. |

### OTHER REFERENCES

1. GuardianRx Patient Care System web-page of Carepoint. www.Carepoint.com
2. “Remote Monitoring of Pulse Oximetry—Improving Patient care” Dec. 19, 2004, by Katherine Sharig
3. “In Hospital Deaths from Medical Errors at 195,000 per Year USA” posted by www.medicalnewstoday.com/articles/11856.php on Aug. 9, 2004 by Scott Shapiro and Sarah Loughran
3. U.S. Code of Federal Regulation 42 CFR Part 483—Federal Minimum Standards of Care
4. Medtronic Remote Monitoring System, Medtronic Inc.
5. Care Trend Monitoring System, Sensitron Inc.
6. “Medical Errors: The Scope of the Problem” by Karen J. Migdail of the Agency for Healthcare Research and Quality, Publication No. AHRQ 00-PO37
7. “Medical Errors Cost U.S. $8.8 Billion, result in 238,337 potentially preventable deaths” a Health Grades study, April 2008, Scott Shapiro, www.healthgrades.com/media/DMS/pdf/HealthGradesPatientSafetyRelease2008.pdf
8. “Medical Errors—A Leading Cause of Death”, Journal of the American Medical Association, Vol 284, No 4, Jul. 26, 2000 by Dr Barbara Starfield, MD, MPH, of the Johns Hopkins School of Hygiene and Public Health
9. “The Impact of Medical errors on Ninety-Day Costs and Outcomes: An Examination of Surgical Patients” by Encinosa, W E, Hellinger F J, Health Services Research, V43(6): 2067-2085
10. “Medication Errors Cost State $17.7 Billion and Cause Harm to 150,000 Californians Annually” a report from a panel created by based on California Senate Concurrent Resolution 49, www.californiaprogressreport.com/2007/03/medication_erro.html, Posted on Mar. 7, 2007
11. “Hospital Medication Errors” by Chris Woolston, Jul. 8, 2003, posted at www.ahealthyme.com/topic/hostpitalmederrors
12. “Fraud found in Medicare billings” By Julie Appleby, USA Today, Mar. 13, 2009

We claim:

1. A patient care monitoring system and method consist of programmable active radio frequency identification devices (RFID) integrated with digital processing, memory and timing circuitry, which will be employed as a patient identification device, as a care giver identification device and as an identification label/tag for each prescribed treatment, procedure, medication and special care action (care action ID tag in short) along with linked sensor networks that determines the patient's laying position, bed wetness, body temperature, pulse rate, heart rate and/or other physiological parameters. Each care action ID tag will be programmed with codes corresponding to the prescribed care action and identification code of the targeted patient. While each patient's identification device will be programmed remotely on daily basis of all the care actions prescribed by his/her physician along with timing for those care actions. Also, each patient's identification device will be programmed to receive sensor signals from the attached (link by wire or wireless) sensor networks to determine the signal type and to properly respond to those signals. At the point-of-care, the care action ID tag will transmit continuously, at a cycle rate of once every second or other frequency rate, signal strings containing its care action identification code and the targeted patient ID code. Upon receiving such signal string by a patient identification device and determining that the string contains its unique patient ID code (the patient ID device will ignore any signal string without containing its own patient ID code), the patient ID device will match care action code in the signal string with its stored care actions program. An “O.K.” signal along with its own patient identification code will be transmitted if there is a match along with activating the visual/audio approval indicator on the patient ID device, or “Mistake” signal with its own patient identification code will be sent if there is not a match along with activating its visual/audio warning indicator. The care action ID tag, upon receiving the “O.K.” or “Mistake” signal string from the patient ID device with matching patient ID code will respond by activating its own visual/audio approval or warning indicator. The care giver will respond accordingly based on the feedback of the patient ID and care action ID devices, thus avoiding errors in administering care actions. At the same time, both the patient ID and the care action ID devices will record this administering and time along with the care givers' ID codes in their memory for transfer to a central processor to be processed into a patient care monitoring report. Equally, the patient's ID device, upon determining a particular response to sensor network signal(s) on a change of status (such as a change of clothing, bed pan and bed sheets after wetness is detected) or based on scheduled care action timing (such as time to alter the laying position), will transmit a signal string with the type of care action required along with the patient identification code to be received by any care givers' ID devices within the patient room to respond. The patient's ID band will also issue such prompt signal string upon determining a “Mistake” in the care action to be administered. This same signal string will also be sent through a relaying device within the patient room to a central processor to forward the alert to the nurse station responsible for the patient to obtain quick response from any care givers at that station. Such prompting signal will be transmitted continuously until the sensor network associated with the said patient ID device provides the signal indicating that proper care action(s) has been administered or a correct signal from a care action ID tag has been received. All the care action administered to the patient will be logged and transferred to the central processor to produce a comprehensive patient care monitoring report to assist quality control for the healthcare facility.

2. The patient's identification device, the care giver's identification device as well as the treatment, procedure, medication and care action identification device specified in claim 1 can be powered by rechargeable battery pack or one-time usage battery pack.

3. The patient's identification device, the care giver's identification device as well as the treatment, procedure, medication and care action identification device specified in claim 1 will be transmitting/receiving in the radio frequency region allocated by the U.S. Federal Commission on Communication for short distance communication without licensing, such as at 2.3 GHz.

4. The patient's identification device described in claim 1 will continuously be in receiving mode between any transmission tasks that it performs, and it will only respond to any RF signal at frequency (such as 2.3 GHz) that it is tuned to and to signal string that contains its own unique identification code.

5. The RF transmission conducted by the care action ID tag, the patient's identification device and the care giver's identification device as described in claim 1 can be in burst mode (microseconds to milliseconds in duration) and at a low power level (in milliwatts range) for only short distance (3-10 feet) effective reception by other identification devices at the point-of-care locations.

6. A relay device located in each patient room as described in claim 1 can be an RF transceiving device that will serve as the relaying device between the central processor and the patient identification devices within the patient room. It will transfer a physician's prescribed care action program, which is entered into the central processor by the patient's physician or care giver, to the corresponding patient's ID device and transfer recorded daily care actions from each patient's ID device to the central processor along with relaying prompt signals issued by the patients' ID bands. This RF transceiving device can be linked to the central processor via wired or wireless intranet such as a local area network (LAN) or wide area network (WAN). It will execute any data format or protocol changes to facilitate transfer via wired or wireless network.

7. The patient's identification device described in claim 1 can further examine its own stored care action program and determine whether a specific care action is overdue. If it determines that such care action is overdue or becoming overdue, it will transmit a coded prompt signal continuously at 1 cycle per second or other frequency rate to alert care giver(s) within the patient room to respond to the prompt, or through the RF transceiving relaying device described in claim 1 to the central processor to alert the responsible nurse station. This type of prompt transmission will be continued until the patient's identification device receives the care action code corresponding to the overdue care action transmitted from the care action tag.

8. The patient's identification device described in claim 1 can be in configuration of a waterproof wrist band, an ankle band, a badge attached to a garment, or a pendant/tag attached to the patient bed.

9. All configurations of a patient's identification device described in claim 1 will each carry an attachment method (from strong adhesive to non-reversible buckles) to the patient that will be difficult to detach once attachment is made.

10. The care giver's identification device described in claim 1 can be in configuration of a waterproof wrist band, integrated with a waterproof wrist watch, an ankle band, a badge attached to garment, or a pendant/badge attached to the care giver.

11. The care giver's identification device described in claim 1 will be in receiving mode in between its transmission bursts. Upon receiving any coded prompt signal from a patient identification device, it will activate the vibration mechanism within and display the care action or its code on the care giver's identification device.

12. The treatment, procedure, medication and special care action identification device described in claim 1 will be in receiving mode in between its transmission burst. Upon receiving any signal from a patient identification device, it will only respond if the patient identification code matches its stored patient identification code.

13. An identification device programming machine will be employed in the system described by claim 1, which can use wireless means to command a care action or a patient identification device placed within its RF shielded compartment to clear its memory and record a new set of identification number.

14. The identification device programming machine described in claim 13 can use electrical contact connection to program an identification device.

15. The identification device programming machine described in claim 13 can be equipped with a printer to print out an attachable label containing associated information at the same time it is programming an identification device with that information. The printed label can then be attached to the identification device either automatically by this programming machine or manually by the person doing the programming.

16. The central processor described in claim 1 can be a computer connected to the central processor of a healthcare facility via intranet or the facility central processor itself. It shall contain the software to translate all prescribed care actions into pre-programmed codes, such as the National Drug Code Directory (NDC), and match the prescribed care actions to assigned patients as well as scheduling. It will then send the data to each individual patient's identification device and to each responsible department and/or nurse station for programming the care action identification devices and for subsequent execution.

17. The central processor described in claim 1 will further gather the care action administration log from each patient's identification device to compose a periodic patient care monitoring report, which will include, but is not limited to, department, any mistakes, overdue care actions, remedy undertook and timing of all care actions along with the patients' as well as care givers' identifications to provide critical data for quality control in patient care of the healthcare facility.

18. The daily and/or periodical patient care monitoring report described in claim 17 can be accessed by government regulatory agencies, healthcare facility accreditation agencies, relevant insurance firms or a patient's immediate family through the Internet or other communication conduits to achieve genuine monitoring of the quality of care provided by a healthcare facility and/or home care provider as well as to the accuracy of medical billings presented by that entity (thus resolving billing disputes and preventing fraud).

19. The patient's identification device, the care giver's identification device as well as the treatment, procedure, medication and care action identification device specified in claim 1 are powered by rechargeable battery pack, then a non-contact electromagnetic charging coil can be used to perform battery charging to maintain the functionality of all those identification devices without the tedium of proper alignment in making electrical contacts.

20. The adoption or the incorporation of the system and method described in claim 1 by entity such as (but not limited to) an acute care, general, mental, and/or specialty hospital, by a nursing care facility, a retirement community and/or a home care provider into its patient care quality control procedure and/or process as well as into its billing accuracy verification procedure.
